(19) United States
(12) Patent Application Publication
(10) Pub. No.: US 2004/0101505 A1
(43) Pub. Date: May 27, 2004

(54) Composition

(75) Inventors: Richard Kevin Payne, Brielle, NJ (US); Nadia Soliman, East Brunswick, NJ (US); Suman Chopra, Dayton, NJ (US); Jean Bakk, Bedminster, NJ (US); Cristina A. Bielli, Hillsborough, NJ (US)

Correspondence Address: Colgate-Palmolive Company, 909 River Road, P.O. Box 1343, Piscataway, NJ, 08855-1343, US

(73) Assignee: Colgate-Palmolive Company

(21) Appl. No.: 10/301,190

(22) Filed: Nov. 21, 2002

(51) Int. Cl. A61K 7/075; A61K 7/08; C11D 17/08

(52) U.S. Cl. 424/70.31; 510/433

(57) ABSTRACT

A composition useful in an area selected from the group consisting of personal care, household surface care and fabric care, which comprises

(a) an active agent in quantities sufficient to bring about a desired effect,

(b) a deposition and/or duration active agent extending amount of a material or materials of the formula.

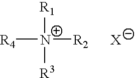

wherein R₁, R₂, R₃ and R₄ are the same or different and are R₅ wherein R₅ is alkyl of one to four carbon atoms, inclusive;

R₆ wherein R₆ is (—CH₂CH₂(CH₂)ₐO—)_{b}R₈ where R₈is hydrogen or methyl, a is 0 or 1 and b is 1, 2, or 3;

R₉ wherein R₉ is a meadowfoam seed oil or seed oil derivative which has a covalent bond with the nitrogen of the formula;

and wherein at least one but no more than two of R₁, R₂, R₃ and R₄ is an R₅;

and wherein at least one but no more than two of R₁, R₂, R₃ and R₄ are an R₆;

and wherein at least one but no more than two of R₁, R₂, R₃ and R₄ are an R₉;

and X is a counterion bearing a negative charge, and

a surface compatible carrier.

### BACKGROUND OF THE INVENTION

Fragrances have been utilized in skin contacting compositions for hundreds of years. Those who use fragrances desire to have an aroma about them that is pleasing to themselves and others in their immediate vicinity. The first criterion of a fragrance in use in a skin contacting composition is that the fragrance be deposited on the skin and stay there for an appropriate time span. Although this can be accomplished relatively easily in “leave on” compositions such as lotions, creams, and the like, it is much more difficult to accomplish when using a rinse off composition using water to remove the composition from the skin after a relatively short contact time with the skin, such as a maximum contact time of about 2 minutes, generally no more than about 90 seconds or about 60 seconds or about 30 seconds. The second criterion is that the fragrance aroma must be releasable from the skin for whatever reason, such as the fragrance vapor pressure, its measure of attachability to the skin, and the like, and at a rate which brings about a significant duration of the aroma at a point detectable above the level of the skin. The ideal fragrance, therefore, is one, which has significant skin substantivity, for purposes of getting it onto the skin, but also manages to have a detectable, desirable aroma above the skin for a significant duration of time. This combination of effects is difficult to achieve with the fragrance per se because the most significant trait of a fragrance is its desirable “aroma” or “scent” not the two variables of concern here. Therefore, a significant amount of time has been put into studying materials, which can improve at least one and preferably two of the above-identified criteria.

A series of materials has been found which significantly increases the substantivity and duration of fragrance aroma. This effect(s) can be significantly increased when a further family of materials is also present. The effects can be present in both leave-on and rise-off compositions used in contacting the skin. Such rinse-off compositions can additionally have a skin cleansing level of a surfactant or mixture of surfactants. Unless in solid form, a substantial amount, preferably a majority amount of the rinse-off compositions, are aqueous.

The benefit of this family(ies) of material can also be effectively applied to other hydrophobic active agents such as antibacterial agents, silicones, anti-fungal agents, and the like. The technology benefits other systems apart from skin care wherein deposition and duration of effect(s) are also desirable such as household surface care products (sinks, toilets, stove tops, floors and counter-tops), and fabric care products such as detergents and softeners. They can be used for the deposition and duration of fragrances, antibacterial agents, odor controlling agents, and the like.

### SUMMARY OF THE INVENTION

In accordance with the invention, there is a composition useful in an area selected from the group consisting of personal care, household surface care, and fabric care, which comprises:

(a) an active agent in quantities sufficient to bring about a desired effect,

(b) a deposition and/or duration extending amount of a material or materials of the formula

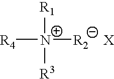

wherein R₁, R₂, R₃ and R₄ are the same or different and are

R₅ wherein R₅ is alkyl of one to four carbon atoms, inclusive;

R₆ wherein R₆ is (—CH₂CH₂(CH₂)ₐO—)_{b}R₈ where R₈ is hydrogen or methyl, a is 0 or 1 and b is 1, 2, or 3;

R₉ wherein R₉ is a meadowfoam seed oil or seed oil derivative which has a covalent bond with the nitrogen of the formula;

and wherein at least one but no more than two of R₁, R₂, R₃ and R₄ is an R₅;

and wherein at least one but no more than two of R₁, R₂, R₃ and R₄ are an R₆;

and wherein at least one but no more than two of R₁, R₂, R₃ and R₄ are an R₉;

and X is a counterion bearing a negative charge, and

(c) a surface compatible carrier.

It has now been found that even more extended deposition and/or duration material, particularly fragrance times, can be achieved with an additional cationic material selected from the group consisting of a cationic polymer, or mixtures thereof..

### DETAILED DESCRIPTION OF THE INVENTION

The usage of component (b) of the invention brings about greater substantivity and duration of time for effectiveness of the fragrance aroma in any composition wherein substantivity and duration of the fragrance aroma are desirable. As disclosed earlier, these compositions are essentially any composition, which has a fragrance wherein the fragrance can be deposited on a surface. Non-limiting examples of these compositions have been provided earlier in this disclosure. Further non-limiting illustrative examples include Hair, clothing, towels, and inanimate surfaces such as floors and counter-tops.

As previously noted, other types of active agents are contemplated beyond fragrances. These active agents' substantivity will also be increased through the use of component (b) of the invention

Generally, these compositions are divided into the compositions, which have a relatively lengthy contact time with a surface and those with relatively short contact time prior to rinsing off the surface, usually with water. Generally, a “leave-on” product which is a personal care lotion or cream is left on the skin for at least about two minutes while a “rinse-off” product is left on the skin for less than about two minutes, preferably no more than about 90, 60, or 30 seconds. Examples of the latter are hand cleansers and body cleansers. The effect of the (b) component is particularly noteworthy with respect to the compositions useful in the personal care area, particularly the skin cleansing, skin care, hair cleansing and hair conditioning, and antiperspirant deodorant product areas. The ability to increase fragrance substantivity and duration of effect in these areas can be very significant both in leave-on and rinse-off compositions. Liquids and gels can be of a viscosity of about 100 to about 10,000 cps or higher. Emollients can be present for skin feel, moisturization and the like. Illustrative examples of such emollients include long chain alkyl or alkenyl fatty acids, fatty acid esters, short chain acids with long chain alcohols such as myristyl propionate, mineral oil, petrolatum, silicone, and the like. Gelling agents such as silicone elastomer (e.g., Dow 9040), silicone polyamides, dibenzylidene sorbitol, xanthan gum and carboxy methylcellulose can be employed. As stated previously, the composition, particularly the liquid and gel, need not be effective for cleansing a surface but can be used to condition a surface, such as for example skin or hair, through conditioning and/or moisturizing. Hair conditioners, skin lotions and creams can be employed. In these skin lotions and creams the compositions can have emulsifying surfactants in small levels because of their emulsifying properties with the various oily materials that make the composition a “lotion” or “cream.”

Of particular significance are compositions wherein there is a surfactant therein and a cleansing of a surface takes effect, for example skin, hard surface, or clothing. In each of these circumstances, the compatible carrier component (c) is usually water or includes water. Some of the carrier include water but are solids such as “soap” bars for cleansing skin and pellets or granules for washing clothing.

Cleansing surfactants which can be employed include: Soap, a long chain alkyl or alkenyl, branched or normal carboxylic acid salt such as sodium, potassium, ammonium or substituted ammonium salt, can be present in the composition as an example of an anionic surfactant. Exemplary of long chain alkyl or alkenyl are from about 8 to about 22 carbon atoms in length, specifically about 10 to about 20 carbon atoms in length, more specifically alkyl and most specifically normal, or normal with little branching. Small quantities of olefinic bond(s) may be present in the predominantly alkyl sections, particularly if the source of the “alkyl” group is obtained from a natural product such as tallow, coconut oil and the like. Because of its potential harshness soap is not a preferred surfactant and can be omitted from the composition.

Other cleansing surfactants can be present in the composition as well. Examples of such surfactants are the anionic, amphoteric, nonionic and cationic surfactants. Examples of anionic surfactants include but are not limited to soaps, alkyl sulfates, anionic acyl sarcosinates, methyl acyl taurates, N-acyl glutamates, acyl isethionates, alkyl sulfosuccinates, alkyl phosphate esters, ethoxylated alkyl phosphate esters, trideceth sulfates, protein condensates, ethoxylated alkyl sulfates and the like.

Alkyl chains for these surfactants are C₈-C₂₂, preferably C₁₀-C₁₈, more preferably C₁₂-C₁₄.

Anionic non-soap surfactants can be exemplified by the alkali metal salts of organic sulfate having in their molecular structure an alkyl radical containing from about 8 to about 22 carbon atoms and a sulfonic acid or sulfuric acid ester radical (included in the term alkyl is the alkyl portion of higher acyl radicals). Preferred are the sodium, ammonium, potassium or triethanolamine alkyl sulfates, especially those obtained by sulfating the higher alcohols (C₈-C₁₈ carbon atoms), sodium coconut oil fatty acid monoglyceride sulfates and sulfonates; sodium or potassium salts of sulfuric acid esters of the reaction product of 1 mole of a higher fatty alcohol (e.g., tallow or coconut oil alcohols) and 1 to 12 moles of ethylene oxide; sodium or potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates; the reaction product of fatty acids having from 10 to 22 carbon atoms esterified with isethionic acid and neutralized with sodium hydroxide; water soluble salts of condensation products of fatty acids with sarcosine; and others known in the art.

Zwitterionic surfactants can be exemplified by those which can be broadly described as derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxy, sulfonate, sulfate, phosphate, or phosphonate. A general formula for these compounds is:

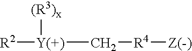

wherein R² contains an alkyl, alkenyl, or hydroxy alkyl radical of from about 8 to about 18 carbon atoms, from 0 to about 10 ethylene oxide moieties and from 0 to I glyceryl moiety; Y is selected from the group consisting of nitrogen, phosphorus, and sulfur atoms; R3 is an alkyl or monohydroxyalkyl group containing 1 to about 3 carbon atoms; X is I when Y is a sulfur atom and 2 when Y is a nitrogen or phosphorus atom, R⁴ is an alkylene or hydroxyalkylene of from 0 to about 4 carbon atoms and Z is a radical selected from the group consisting of carboxylate, sulfonate, sulfate, phosphonate, and phosphate groups.

Examples include: 4-[N,N-di(2-hydroxyethyl)-N-octadecylammonio]-butane-1-carboxylate; 5-[S-3-hydroxypropyl-S-hexadecylsulfonio]-3 hydroxypentane-1-sulfate; 3-[P,P—P-diethyl-P 3,6,9 trioxatetradecyl-phosphonio]-2-hydroxypropane-1-phosphate; 3-[N,N-dipropyl-N-3 dodecoxy-2-hydroxypropylammonio]-propane-I-phosphonate; 3-(N,N-di-methyl-N-hexadecylammonio)propane-1-sulfonate; 3-(N,N-dimethyl-N-hexadecylammonio)-2-hydroxypropane-1-sulfonate; 4-(N,N-di(2-hydroxyethyl)-N-(2 hydroxydodecyl)ammonio]-butane-1-carboxylate; 3-[S-ethyl-S-(3-dodecoxy-2-hydroxypropyl)sulfonio]-propane-1-phosphate; 3-(P,P-dimethyl-P-dodecylphosphonio)-propane-1-phosphonate; and 5-[N,N-di(3-hydroxypropyl)-N-hexadecylammonio]-2-hydroxy-pentane-1-sulfate.

Examples of amphoteric surfactants which can be used in the compositions of the present invention are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecylaminopropionate, sodium 3-dodecylaminopropane sulfonate, N-alkyltaurines, such as the one prepared by reacting dodecylamine with sodium isethionate according to the teaching of U.S. Pat. No.2,658,072, N-higher alkyl aspartic acids, such as those produced according to the teaching of U.S. Pat. No. 2,438,091, and the products sold under the trade name “Miranol” and described in U.S. Pat. No. 2,528,378. Other amphoterics such as betaines are also useful in the present composition.

Examples of betaines useful herein include the high alkyl betaines such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydro-xypropyl)alpha-carboxyethyl betaine, etc. The sulfobetaines may be represented by coco dimethyl sulfopropyl betaine, stearyl dimethyl sulfopropyl betaine, amido betaines, amidosulfobetaines, and the like.

Many cationic surfactants are known to the art. By way of example, the following may be mentioned:

stearyldimenthylbenzyl ammonium chloride;

dodecyltrimethylammonium chloride;

nonylbenzylethyldimethyl ammonium nitrate;

tetradecylpyridinium bromide;

laurylpyridinium chloride;

cetylpyridinium chloride

laurylpyridinium chloride;

laurylisoquinolium bromide;

ditallow(Hydrogenated)dimethyl ammonium chloride;

dilauryldimethyl ammonium chloride; and

stearalkonium chloride.

Additional cationic surfactants are disclosed in U.S. Pat. No. 4,303,543 see column 4, lines 58 and column 5, lines 1-42, incorporated herein by references. Also see CTFA Cosmetic Ingredient Dictionary, 6th Edition 1995, pages 795-799 for various long chain alkyl cationic surfactants; incorporated herein by references.

Nonionic surfactants include those which can be broadly defined as compounds produced by the condensation of alkylene oxide groups (hydrophilic in nature) with an organic hydrophobic compound, which may be aliphatic or alkyl aromatic in nature. Examples of preferred classes of nonionic surfactants are:

1. The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 10 to 60 moles of ethylene oxide per mole of alkyl phenol. The alkyl substituent in such compounds may be derived from polymerized propylene, diisobutylene, octane, or nonane, for example.

2. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.

3. The condensation product of aliphatic alcohols having from 8 to 18 carbon atoms, in either straight chain or branched chain configuration with ethylene oxide, e.g., a coconut alcohol ethylene oxide condensate having from 10 to 30 moles of ethylene oxide per mole of coconut alcohol, the coconut alcohol fraction having from 10 to 14 carbon atoms. Other ethylene oxide condensation products are ethoxylated fatty acid esters of polyhydric alcohols (e.g., Tween 20-polyoxyethylene (20) sorbitan monolaurate).

4. Long chain tertiary amine oxides corresponding to the following general formula:

R₁R₂R₃N→O

wherein R₁ contains an alkyl, alkenyl or monohydroxy alkyl radical of from about 8 to about 18 carbon atoms, from 0 to about 10 ethylene oxide moieties, and from 0 to 1 glyceryl moiety, and, R₂ and R₃ contain from 1 to about 3 carbon atoms and from 0 to about 1 hydroxy group, e.g., methyl, ethyl, propyl, hydroxy ethyl, or hydroxy propyl radicals. The arrow in the formula is a conventional representation of a semipolar bond. Examples of amine oxides suitable for use in this invention include dimethyidodecylamine oxide, oleyl-di(2-hydroxyethyl)amine oxide, dimethyloctylamine oxide, dimethyldecylamine oxide, dimethyl-tetradecylamine oxide, 3,6,9 trioxaheptadecyidiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethyl-hexadecylamine oxide.

5. Long chain tertiary phosphine oxides corresponding to the following general formula:

RR′R″P→O

wherein R contains an alkyl, alkenyl or monohydroxyalkyl radical ranging from 8 to 20 carbon atoms in chain length, from 0 to about 10 ethylene oxide moieties and from 0 to 1 glyceryl moiety and R′ and R″ are each alkyl or mono-hydroxyalkyl groups containing from 1 to 3 carbon atoms. The arrow in the formula is a conventional representation of a semipolar bond. Examples of suitable phosphine oxides are: dodecyidimethylphosphine oxide, tetra-decylmethylethylphosphine oxide, 3,6,9-trioxaoctadecyidimethylphosphine oxide, cetyldimethylphosphine oxide, 3-dodecoxy-2-hydroxypropyldi(2-hydroxyethyl)phosphine oxide stearyldimethylphosphine oxide, cetylethyl propylphosphine oxide, oleyldiethylphosphine oxide, dodecyidiethylphosphine oxide, tetra-decyldiethylphosphine oxide, dodecyidipropylphosphine oxide, dodecyl-di(hydroxymethyl)phosphine oxide, dodecyldi(2-hydroxyethyl)phosphine oxide, tetradecylmethyl-2-hydroxypropylphosphine oxide, oleyldimethylphosphine oxide, 2-hydroxydodecyldimethylphosphine oxide.

6. Long chain dialkyl sulfoxides containing one short chain alkyl or hydroxy alkyl radical of 1 to about 3 carbon atoms (usually methyl) and one long hydrophobic chain which contain alkyl, alkenyl, hydroxy alkyl, or keto alkyl radicals containing from about 8 to about 20 carbon atoms, from 0 to about 10 ethylene oxide moieties and from 0 to 1 glyceryl moiety. Examples include: octadecyl methyl sulfoxide, 2-ketotridecyl methyl sulfoxide, 3,6,9-trioxaoctadecyl 2-hydroxyethyl sulfoxide, dodecyl methyl sulfoxide, oleyl 3-hydroxypropyl sulfoxide, tetradecyl methyl sulfoxide, 3 methoxytridecylmethyl sulfoxide, 3-hydroxytridecyl methyl sulfoxide, 3-hydroxy-4-dodecoxybutyl methyl sulfoxide.

7. Alkylated polyglycosides wherein the alkyl group is from about 8 to about 20 carbon atoms, preferably about 10 to about 18 carbon atoms and the degree of polymerization of the glycoside is from about 1 to about 3, preferably about 1.3 to about 2.0.

Fragrances include any materials which provide an aroma when on a surface. These are generally due to the volatile perfume ingredients therein.

The volatile perfume ingredients employed in the personal cleansing, hard surface cleansing and fabric cleansing compositions of the present invention are the conventional ones known in the art. Selection of the perfume ingredients used in the liquid personal cleansing shower gel compositions of the present invention can be based on the desired fragrance characteristics for the composition.

Suitable perfume compounds and compositions can be found in the art including U.S. Pat. No. 4,145,184, Brain and Cummins, issued Mar. 20, 1979; U.S. Pat. No. 4,209,417, Whyte, issued Jun. 24, 1980; U.S. Pat. No. 4,515,705, Moeddel, issued May 7, 1985; and U.S. Pat. No. 4,152,272, Young, issued May 1, 1979, all of said patents being incorporated herein by reference.

Perfumes can be classified according to their volatility. For purposes of the present invention, “volatile” perfumes are those having a boiling point of less than about 500° C. The highly volatile, low boiling, perfume ingredients typically have boiling points of about 250° C. or lower. The moderately volatile perfume ingredients are those having boiling points of from about 250° C. to about 300° C. The less volatile, high boiling, perfume ingredients are those having boiling points of about from about 300° C. to about 500° C. Many of the perfume ingredients as discussed hereinafter, along with their odor and/or flavor characters, and their physical and chemical properties, such as boiling point and molecular weight, are given in “Perfume and Flavor Chemicals (Aroma Chemicals),” Steffen Arctander, published by the author, 1969, incorporated herein by reference. It is preferred that the products herein particularly the liquid or gel personal cleansing products herein contain, as measured by the total fragrance, at least about 5%, more preferably about 25%, and most preferably at least about 50% of highly volatile perfume ingredients having a boiling point of 250° c. or lower.

Examples of the highly volatile, low boiling, perfume ingredients are: anethole, benzaldehyde, benzyl acetate, benzyl alcohol, benzyl formate, iso-bornyl acetate, camphene, cis-citral (neral), citronellal, citronellol, citronellyl acetate, para-cymeme, dicenal, dihyrolinalool, dihydromyrcenol, dimethyl phenyl carbinol, eucalyptol, geranial, geraniol, geranyl acetate, geranyl nitrile, cis-3-hexenyl acetate, hydroxycitronellal, di-limonene, linalool, linalool oxide linalyl acetate, linalyl propionate, methyl anthranilate, alpha-methyl ionone, methyl nonyl acetaldehyde, methyl phenyl carbinyl acetate, laevomenthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, and vertenex (para-tertiary-butyl cyclohexyl acetate). Some natural oils also contain large percentages of highly volatile perfume ingredients. For example, lavandin contains as major components: linalool; linalyl acetate; geraniol; and citronellol. Lemon oil and orange terpenes both contain about 95% of d-limonene.

Examples of moderately volatile perfume ingredients are: amyl cinnamic aldehyde, iso-amyl salicylate, beta-caryophylene, cedrene, cinnamic alcohol, coumarin, dimethyl benzyl carbinyl acetate, ethyl vanillin, eugenol, iso-eugenol, flor acetate, heliotropine, 3-cis-hexenyl salicylate, hexyl salicylate, lilial (para-tertiarybutyl-alpha-methyl hydrocinnamic aldehyde), gamma-methyl ionone, nerolidol, patchouli alcohol, phenyl hexanol, beta-selicarb, trichloromethyl phenyl carbinyl acetate, triethyl citrate, vanillin, and veratraldehyde. Cedarwood terpenes are composed mainly of alpha-cedrene, beta-cedrene, and other C₁₅H₂₄ sesquiterpenes.

Examples of the less volatile, high boiling, perfume ingredients are: benzophenone, benzyl salicylate, ethylene brassylate, galaxolide (1,3,4,6,7,8-hexahydro-4,6,6,7,8,8-hexamethyl-cyclopenta-gama-2-benzopyran)hexyl cinnamic aldehyde, lyral(4-(4-hydroxy4-methyl pentyl)-3-cyclohexene-10-carboxaldehyde), methyl cedrylone, methyl dihydro jasmonate, methyl-beta-naphthyl ketone, musk indanone, musk ketone, musk tibetene, and phenylethyl phenyl acetate.

The material that enhances the substantivity and duration of the fragrance is meadowfoam seed oil or any derivative thereof that is capable of forming a covalent bond with the nitrogen of component (b) of the formula. Meadowfoam seed oil and its derivatives are particularly able in promoting deposition. The meadowfoam seed oil is commonly found in native state in Limanthes Alba as the triglyceride with fatty acids being a mix of alkenoic fatty acids, mostly C₂₀, i.e., 5-eicosenoic acid, others being C₂₁ i.e., 5,13-heneicosenoic acid while still others being a mix of C₂₂, i.e., 5-docosenoic acid and C₂₂, i.e. 13-docosenoic acid.

Various issued patents have disclosed the meadowfoam seed oil constituents and its derivatives in a more detailed manner, e.g. U.S. Pat. Nos. 5,646,321; 5,741,915; 5,741,916; and 5,741,919 incorporated by reference wherein the following structure and quantities are mentioned.

The fatty distribution of the oil ranges from 20 to 22 carbons and has unsaturation in specific locations. The oil contains 97% by weight higher unsaturated alkyl groups. Typically, meadowfoam oil is believed to contain 60-65% of a twenty carbon terminal mono-carboxy acid having one unsaturation between carbon 5 and 6. Additionally, it contains 12-20% of a twenty two carbon terminal mono-carboxy acid having one unsaturation between carbon 5 and 6, and 15-28% of a twenty two carbon terminal mono-carboxy acid having one unsaturation between carbon 5 and 6, an another between carbon 13 and 14. These are shown below structurally.

60-65% by weight

HOOC—(CH₂)₃—CH═CH—(CH₂)₁₃—CH₃

12-20% by weight a mixture of

HOOC—(CH₂)₃—CH═CH—(CH₂)₁₅—CH₃

and

HOOC—(CH₂)₁₁—CH═CH—(CH₂)₇—CH₃

and

15-28% by weight

HOOC—(CH₂)₃—CH═CH—(CH₂)₆—CH═CH—(CH₂)₆—CH₃

Meadowfoam seed oil can also be present in its glyceride form.

Derivatives of the meadowfoam oil carboxylic acids can be used in the compositions of this invention as well as the long chain carboxylic acids depicted above. These involve performing standard chemistry on the carboxy group while maintaining the unsaturation untouched or at least essentially untouched. Examples of such derivatives are the esters, for example, as disclosed in U.S. Pat. Nos. 5,741,919 and 5,646,321; the alkanolamides, for examples, as disclosed in U.S. Pat. No. 5,741,916 and the betaines derivatives, for example, as disclosed in U.S. Pat. No. 5,741,915. Additionally, a derivative of meadowfoam seed oil includes materials wherein the normal distribution of the meadowfoam oil can be altered to accentuate the quantity of one or more of the unsaturated alkyl monocarboxylic acids. For example, the total concentration of erucic acid, a C₂₂ unsaturated monocarboxylic acid can be enriched to 80 or more wt. percent of the meadowfoam oil composition. These quaternary ammonium materials based on meadowfoam oils are available from The Fanning Corporation, 2450 West Hubbard Street, Chicago, Ill. 60612. Further exemplification is an alkenyl grouping, which can be readily prepared. All these materials can be covalently bonded to the quaternary nitrogen atom of component (b). A specific example of a component (b) material is available from Fanning as Meadowquat®HG. Its structure is

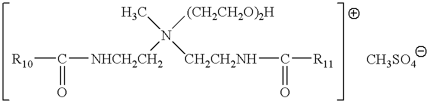

With reference to schematic formula of component (b), X is methosulfate, R₁ is methyl, R₂ is ethoxy (wherein a is zero, b is 2, and R₈ is hydrogen, and R₁₀ and R₁₁ are the same and are the long chain hydrocarbon substituent of a meadowfoam seed oil which is bonded to an amido ethyl group wherein the last carbon at the ethyl is attached to the quaternary nitrogen. This molecule marketed by Fanning is known Meadowquat®HG also known in the INCI nomenclature as PEG-2 dimeadowfoam amido ethyl imonium methosulfate,

The meadowfoam oil carboxylic acids or derivatives thereof can be used in the composition at deposition enhancing quantities, generally a minimum of about 0.25 wt %, 0.5 wt % or about 1.0 or 2.0 wt %. A maximum quantity is generally no higher than about 6 wt %, or preferably about 5, 4 or 3 wt % of the composition.

The counterion of the quaternary ammonium material is any anionic substance such as chloride, nitrate, sulfate, phosphate, methosulfate and the like.

The effectiveness of the quaternary ammonium material in increasing substantivity and duration of a fragrance is significantly enhanced by the presence of a second cationic material, which is a cationic polymer. Examples of such cationic materials include the various polyquats known to the art which include but are not limited to Polyquaternium 2 (a polyelectrolyte formed from quaternized ioenes), Polyquaternium 4 (hydroxycellulose diallyldimethyl ammonium chloride), Polyquaternium 5 (acrylamide/β-methacryloxyethyltrimethyl ammonium methosulfate), Polyquaternium 6 and 7 (homopolymer of dimethyl diallyl ammonium chloride and the copolymer of dimethyl diallyl ammonium chloride with acrylamide), Polyquaternium 8 (methyl and stearyl dimethylaminoethyl methacrylate quaternized with dimethylsulfate), Polyquaternium 10 (1-hydroxypropyl trimethyl ammonium chloride ethers of hydroxyethyl cellulose), Polyquaternium 11 (quaternized PVP and dimethylaminoethyl methacrylate), Polyquaternium 16 (copolymer of PVP/methyl vinylimidazoline), Polyquaternium 17 and 18 (polyelectrolyte formed from quaternized ioenes), Polyquaternium 19 (a vinyl alcohol hydroxypropyl amine salt), Polyquaternium 24 (polymeric quaternized ammonium salt of hydroxymethylcellulose and lauryl dimethyl ammonium substituted epoxide), Polyquaternium 27 (polyelectrolyte formed from quaternized ioenes). Other cationic materials include Polycare 133 (a polymethyacrylamideopropyl trimonium chloride from Rhone-Poulenc). These materials are known to be the type that neutralizes a negative charge, such as on skin.

The physical form of the composition can be a solid, liquid or gel. A solid can illustratively be in the form of powder, granule, bar, precipitated on a carrier such as a fabric softener backing and the like.

When employed in a composition of the invention that requires a cleansing action on a surface, the amount of surfactant or mixture of surfactants is a cleansing effective amount. Depending upon the surfaces to be cleansed and the physical form of the composition, a minimum of about 1,2,3,4,5 or 6 wt. % of surfactant can be employed in the liquid or gel composition. Generally, for liquids or gels the maximum amount of surfactants is about 5 to 40 wt. %, preferably about 25, 20, or 15 wt. % of the composition. For solids, such as bars, granules and the like, a higher maximum and minimum of surfactant is employed. Generally, a minimum of about 40 wt. % surfactant in the composition is employed, preferably about 45, 50, 55, or 60 wt. %. The maximum amount of surfactant employed in the solid is generally no more than about 90 wt. %, preferably no more than about 85,80, or 75 wt. % of the composition. The amount of water in a cleansing liquid or gel is a minimum of about 40 wt. % of the composition, preferably at least about 50, 60 or 70 wt. %. For a solid such as a cleansing bar, a minimum of about 5 wt. % of the composition, desirably about 7, 10 or 15 wt. % is water.

The total amount of volatile perfume present is enough to provide an aroma. Generally, a minimum of about 0.01 preferably 0.05, 0.1, 0.2, or 0.4 wt. % of the composition can be employed. Generally, no more than about 3 wt. %, preferably about 2, 1 or 0.5 wt. % of the composition is employed.

The additional cationic deposition material when it is present can be a minimum of about 0.05, 0.1, or 0.2 wt. % of the composition. The maximum is generally no more than about 1.0 or even less about 0.8, 0.6, or 0.5 wt. % of the composition.

The compositions of the invention are prepared by standard methods generally known in the art. In order to ensure proper stability and performance of the composition with respect to deposition and/or duration of the agent, specifically fragrance, it is preferred to add the component (b) material to the composition prior to the fragrance or other active agent and the cationic polymer, assuming the cationic polymer is used, which is preferable.

When using the component (b) material enhanced deposition on a surface and/or duration of activity of the fragrance or other active agent is observed. With the presence of the additional cationic polymer, the results can be even more pronounced. Dramatic increases in deposition (substantivity) to the surface by the active agent such as a fragrance, antimicrobial, anti-fungal and emollients can be obtained and/or duration of activity.

The results are assessed using an in vitro test, which has been validated by trained test human panels for the fragrance testing.

### Analytical Methodology

The in vitro methodology employs the use of a wool (worsted fabric) swatch that is washed in the test product solution, rinsed I a 100-ppm total hardness (Ca⁺²/Mg⁺²) water and then blotted dry with a paper towel. The swatch is then transferred to a headspace-sampling flask that is maintained at a constant temperature of 32° C., which is recognized as average skin temperature. A tube containing a polymer adsorbent (Tenax™ TA (2,6-diphenylene oxide polymer) 60/80 mesh) is used to capture volatile fragrance materials exiting the sampling flask. The wool swatch is purged with 99.99% pure Nitrogen at a flow rate of 50 mL/min which is equivalent to a total volume of 3 liters. At the end of one hour, the purge flow is stopped and the tube is removed and capped. A fresh, pre-conditioned tube is reconnected to the sample flask and the purging procedure is repeated until five tubes (over five hours) are collected. The tubes are then subject to thermal desorption using an Automated Thermal Desorber (ATD) which is interfaced to a GC column (inside the GC oven) via a fused silica transfer line. Fragrance components are separated by the GC column and detected on an Ion Trap Mass Spectrometer and peak areas are integrated by the controlling computer system. Peak area data for each sample (or time point) are converted to ASCII files and process to yield a table with all fragrance components collected over the five hour period. Only the peak areas of fragrance components are totaled (summed) for each sample time and plotted against time.

Validation studies with human test panels using a fragrance(s) in bar soap and shower gel confirmed the validity of the in vitro test method.

Below are experiments showing the same formulations with and without specific quantities of Meadowquat HG® (PEG-2 Dimeadow foam amido ethylmonium Methosulfate) (CTFA name) available from Fanning Corporation. An additional sample had this meadowfoam seed oil derivative and 0.2 wt. % of the composition of the cationic deposition polymer Polyquat 7. The surfactant containing composition (shower gel base) used in the experiments below is as follows:

TABLE 1
Shower Gel Base

| Component | wt/wt % |
|---|---|
| Sodium laureth sulfate | 8.20 |
| Cocamidopropyl betaine | 3.00 |
| Alkyl polyglucoside | 1.12 |
| Fragrance | 1.00 |
| Preservatives | 0.30 |
| Citric acid | 0.06 |
| Water, salt and cationic material | Q.S |
| Total | 100.00 |

Experiments:

| | |
|---|---|
| Control | 0% Meadowquat HG/0% Polyquaternium-7 |
| Exp. 1 | 1% Meadowquat HG/0% Polyquaternium-7 |
| Exp. 2 | 2% Meadowquat HG/0% Polyquaternium-7 |
| Exp. 3 | 3% Meadowquat HG/0% Polyquaternium-7 |
| Exp. 4 | 2% Meadowquat HG/0.20% Polyquaternium-7 |
| | (Non preferred order of addition) |
| Exp. 5 | 1% Meadowquat HG/0.20% Polyquaternium-7 |
| | (Preferred order of addition). |

### Results

The base formula given above was prepared by adding the anionic, amphoteric and nonionic surfactants to water, which were mixed until clear. Preservatives were added until clear then the pH of the preparation was adjusted to between 5.0 and 6.5 using aqueous citric acid. In the case of the control formula, the fragrance was then added and mixed until clear, after which the formula viscosity was adjusted to between 4000 and 10000 cps (centipoise) using anhydrous sodium chloride. The formula was finally Q.S with water to 100 wt/wt %

Experimental formulas 1-3 were prepared as above except the cationic material (Meadowquat HG) and fragrance were added together and allowed to mix until clear. The viscosity was then adjusted as above and the formula Q.S. with water to 100 wt/wt %.

Experimental formula 4 was prepared as above for experimental formulas 1-3 except the combined cationic material (Polyquaternium 7/Meadowquat HG) and fragrance were added together and allowed to mix until clear. The viscosity was then adjusted as above and the formula Q.S. with water to 100 wt/wt %.

Experimental formula 5 was prepared as in the shower gel base, except the cationic materials and fragrance were added in a specific order and allowed to mix until clear. First the Meadowquat HG is added to the shower gel base and mixed until the product is completely clear. This process was repeated for the fragrance and then the Polyquaternium 7. It is important that the Meadowquat HG be added prior to the fragrance and Polyquaternium 7 so as to achieve enhanced phase stability. After addition of cationic material and fragrance, the viscosity was then adjusted as above and the formula Q.S. with water to 100 wt/wt %.

The fragrance longevity measured for the experimental formulas 2-5 is put forth in Table 1. Here the percent increase in total peak area for an experimental formula (relative to the control formula) has been calculated.

TABLE 2
% Increase in fracirance intensity measured
as integrated peak areas for experimental
formulas versus control formula

| Experimental Formula | 1 hr | 2 hr | 3 hr | 4 hr | 5 hr |
|---|---|---|---|---|---|
| Exp. 1 (1% Meadowquat HG/ | 29 | 160 | 96 | 102 | 90 |
| 0% Polyquaternium-7) | | | | | |
| Exp. 2 (2% Meadowquat HG/ | 95 | 235 | 266 | 312 | 307 |
| 0% Polyquaternium-7) | | | | | |
| Exp. 3 (3% Meadowquat HG/ | 188 | 278 | 187 | 164 | 193 |
| 0% Polyquaternium-7) | | | | | |
| Exp. 4 (2% Meadowquat HG/ | 144 | 536 | 2647 | 4100 | 3887 |
| 0.20% Polyquaternium-7) | | | | | |
| Exp. 5 (1% Meadowquat HG/ | −13 | 190 | 344 | 599 | 526 |
| 0.20% Polyquaternium-7 | | | | | |

Addition of Meadowquat HG to the base formula shows an increase in fragrance intensity over time, relative to the control formula (Table 2). The addition of a second cationic material, Polyquaternium 7, to the 2% Meadowquat HG formula (Experimental formula 4) provides a significant percent increase in fragrance intensity compared to Experimental formula 3 (without Polyquaternium 7). Thermal and freeze/thaw stability of experimental formulas 1-4 showed some flocculation after three to six weeks at 120° F. and four weeks after freeze/thaw cycling at 32° F. to 72° F.

Experimental formula 5 was prepared with a lower level of cationic material (1% Meadowquat HG /0.2% Polyquaternium 7) with a different process of preparation, the preferred process as noted above. Experimental formula 5 was found to be stable after 13 weeks at 120° F. and after 13 weeks of freeze/thaw cycling from 32° F. to 72° F. The percent increase in fragrance intensity for this formula ranged from 190% at 2 hours to 600% after 4 hours.

Illustrated below are various other liquid formulations wherein an active agent such as a antimicrobial or fragrance is present with a meadowfoam seed oil quaternary material and preferably a cationic deposition polymer to bring about increased deposition and/or duration of activity of the active agent.

TABLE 3
Liquid Hand Soap

| Ingredient | Wt/Wt % |
|---|---|
| Water | 74.85 |
| PEG Glyceryl monococoate | 1.5 |
| Sodium Laureth Sulfate (2 EO) | 5.37 |
| Sodium Lauryl Sulfate | 10.00 |
| Cocoamidopropyl Betaine | 5.00 |
| Alkyl polyglucoside | 1.13 |
| Triclosan | 0.15 |
| Polyquaternium-7 | 0.2 |
| Meadowquat HG (Fanning) | 1.0 |
| Fragrance | 1.0 |
| DMDM Hydantoin | 0.4 |
| Citric Acid-anhydrous | 0.1 |

TABLE 4
Liquid Body Wash

| Ingredient | Wt/Wt % |
|---|---|
| Water | 83.14 |
| Sodium Laureth Sulfate | 8.20 |
| Cocamidopropyl Betaine | 3.00 |
| Sodium Chloride | 1.44 |
| Alkyl Polyglucoside | 1.13 |
| Meadowquat HG | 1.00 |
| Fragrance | 1.00 |
| Citric Acid | 0.60 |
| Preservatives | 0.29 |
| Polyquaternium 7 | 0.20 |

TABLE 5
Liquid Detergent

| Ingredient | Wt/Wt % |
|---|---|
| Water | 70.98 |
| Dodecylbenzene Sulfonate | 12.36 |
| Ethoxylated C₁₂-C₁₄ Alcohol | 9.50 |
| Silicate | 4.56 |
| Meadowquat HG | 1.00 |
| Preservative | 0.77 |
| Perfume | 0.50 |
| Polyquaternium 7 | 0.20 |
| Color | 0.13 |

TABLE 6
Dish Washing Liquid

| Ingredient | wt/wt % |
|---|---|
| Water | 57.89 |
| Ethoxylated alcohol sulfate sodium salt | 25.86 |
| Dodecylbenzene sulfonate magnesium salt | 6.30 |
| Alkyl Polyglucoside | 3.00 |
| Laurylmonomethyl ethanolamine/ | 2.56 |
| Sodium Xylene Sulfonate | |
| Sodium Chloride | 1.99 |
| Meadowquat HG | 1.00 |
| Magesium Sulfate | 0.50 |
| Color | 0.30 |
| Perfume | 0.25 |
| Polyquaternium-7 | 0.20 |
| Preservative | 0.153 |

1. A composition useful in an area selected from the group consisting of personal care, household surface care and fabric care, which comprises
(a) an active agent in quantities sufficient to bring about a desired effect,
(b) a deposition and/or duration active agent extending amount of a material or materials of the formula: 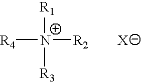
wherein R₁, R₂, R₃ and R₄ are the same or different and are R₅ wherein R₅ is alkyl of one to four carbon atoms, inclusive;
R₆ wherein R₆ is (—CH₂CH₂(CH₂)ₐO—)_{b}R_{b}R₈ where R₈ is hydrogen or methyl, a is 0 or 1 and b is 1, 2, or3;
R₉ wherein R₉ is a meadowfoam seed oil or seed oil derivative which has a covalent bond with the nitrogen of the formula;
and wherein at least one but no more than two of R₁, R₂, R₃ and R₄ is an R₅;
and wherein at least one but no more than two of R₁, R₂, R₃ and R₄ are an R₆;
and wherein at least one but no more than two of R₁, R₂, R₃ and R₄ are an R₉;
and X is a counterion bearing a negative charge, and
a surface compatible carrier.

2. The composition in accordance with claim 1 wherein the area is personal care.

3. The composition in accordance with claim 1 wherein the area is household surface care.

4. The composition in accordance with claim 1 wherein the area is fabric care.

5. The composition in accordance with claim 2 wherein the active agent is a fragrance.

6. The composition in accordance with claim 3 wherein the active agent is a fragrance.

7. The composition in accordance with claim 4 wherein the active agent is a fragrance.

8. The composition in accordance with claim 5 wherein a cleansing amount of a surfactant or mixture of surfactant is present.

9. The composition in accordance with claim 8 wherein the composition is a solid.

10. The composition in accordance with claim 8 wherein the composition is a liquid or gel.

11. The composition in accordance with claim 9 wherein about 0.25 to about 4.0 wt. % of the composition is component (b).

12. The composition in accordance with claim 10 wherein about 0.25 to about 4.0 wt. % of a composition is component (b).

13. The composition in accordance with claim 11 wherein additionally present is a deposition effective amount of a cationic polymer, mixtures of polymer.

14. The composition in accordance with claim 12 wherein additionally present is a deposition effective amount of a cationic polymer, mixtures of polymer.

15. The composition in accordance with claim 13 wherein from 0.01 to about 1.0 wt. % of the composition is the cationic polymer, or mixtures of polymer.

16. The composition in accordance with claim 14 wherein from 0.01 to about 1.0 wt. % of the composition is the cationic polymer, or mixtures of polymer.
